(19) United States
(12) Patent Application Publication
(10) Pub. No.: US 2025/0000121 A1
(43) Pub. Date: Jan. 2, 2025

(54) METHOD FOR REDUCING THE BITTERNESS OF A LEGUMINOUS PROTEIN

(71) Applicant: ROQUETTE FRERES, Lestrem (FR)

(72) Inventors: Christophe LAROCHE, FOUGERES (FR); Aline LECOCQ, MOUVAUX (FR); Guillaume FARDIN, LILLE (FR)

(21) Appl. No.: 18/694,637

(22) PCT Filed: Sep. 21, 2022
(86) PCT No.: PCT/EP2022/025437

§ 371 Date: Mar. 22, 2024

(30) Foreign Application Priority Data
Sep. 24, 2021 (FR) 2110088

(51) Int. Cl. A23J 3/34; A23J 1/14; A23J 3/14; C07K 1/14

(52) U.S. Cl. CPC A23J 3/346; A23J 1/14; A23J 1/148; A23J 3/14; C07K 1/145

(57) ABSTRACT

The invention relates to a method for reducing the bitterness of a legume protein during an extraction process, said extraction process comprising treatment with a phytic acid degrading enzyme.

### TECHNICAL FIELD

The invention relates to the field of legume proteins, in particular of legume protein isolates, even more particularly of pea protein isolates. The invention relates in particular to a method for reducing the bitterness of a legume protein.

### PRIOR ART

Human daily requirements for proteins are between 12 and 20% of food intake. These proteins are provided equally by products of animal origin (meat, fish, eggs, dairy products) and by plant-based food (cereals, leguminous plants, seaweed).

However, in developed countries, protein intake is predominantly in the form of proteins of animal origin. And yet, numerous studies show that excessive consumption of proteins of animal origin to the detriment of plant proteins is one of the causes of increases in cancer and cardiovascular diseases.

Moreover, animal proteins have many drawbacks, both in terms of their allergenicity, notably proteins from milk or eggs, and in environmental terms, in connection with the harmful effects of intensive farming.

Thus, there is an increasing demand from manufacturers for compounds of plant origin having beneficial nutritional and functional properties without, however, having the disadvantages of compounds of animal origin.

Since the 1970s, the development of pulse plants, in particular including pea, in Europe and mainly in France, has dramatically increased as an alternative protein resource to animal proteins for animal and human food consumption. The term “pea” is considered here in its broadest accepted use and includes, in particular, all the wild varieties of “smooth pea” and all the mutant varieties of “smooth pea” and “wrinkled pea”, regardless of the uses for which said varieties are usually intended (human food, animal feed and/or other uses). These seeds are generally non-GMOs and do not require a de-oiling step using solvents.

The pea contains approximately 27% by weight of protein substances. Pea protein, predominantly pea globulin, has been extracted and utilized industrially for a great number of years. Mention may be made, as an example of a method for extracting pea protein, of patent EP1400537. In this process, the seed is milled in the absence of water (process referred to as “dry milling”) in order to obtain a flour. This flour will then be suspended in water in order to extract the protein therefrom.

Legume proteins, and in particular pea proteins, however, suffer from a variable organoleptic quality, since legume proteins frequently generate astringency or unpleasant flavors such as a beany, earthy or bitter taste.

Bitterness is one of the five main flavors, with the sweet, salty, acidic and umami. This flavor, generally considered to be harsh and unpleasant, is the characteristic flavor of products such as quinine, brucine and caffeine. It is created by certain substances that bind with the receptors specific to bitterness on the taste buds on the tongue, and which transmit a signal to the brain through a neurochemical mechanism.

It is thus also possible to distinguish the flavors of smells, which flavors have their own specific and distinct mechanisms.

Users of these legume proteins are aware of these flavor issues and have developed formulation strategies based mainly on the use of flavorings, but also on the use of salts.

One example that can be cited is patent application WO 2019/048564 from RHODIA, which teaches that the use of a vanilla flavoring reduces the bitter taste in a finished food product such as a high-protein drink or cream.

Document WO2021/100729 discloses the use of an agent for suppressing an abnormal flavor, and in particular bitterness, which is a cyclic compound. Legume proteins are in particular cited as ingredients whose bitterness is reduced when mixed with this agent.

Document WO2020/240144 in the name of the Applicant discloses a particular process for manufacturing a protein co-atomized with a flavoring. The bitterness of the protein thus obtained is reduced, thus allowing its use in all known food applications.

Another solution has been disclosed in WO2019/048804, also in the name of the Applicant, to obtain a food product based on legume proteins, for example a ready-to-drink beverage, with improved organoleptic properties. This solution is based on a particular process for manufacturing the food product using a selected compound: sodium citrate. The bitterness of the food product thus obtained is reduced.

A disadvantage related to the use of a flavoring or a masking agent is the need to label this compound on the protein ingredient. Moreover, this requires perfectly controlling the flavoring dosage in the protein in order to achieve the desired improvement without “denaturing” the taste of the protein, that is, without imparting a non-natural taste, for example vanilla, to the pea protein.

The use of salt during the manufacture of a food product for its part increases the mineral content in the finished food product, which may be unacceptable for certain uses.

All of these solutions involve adding an additional ingredient to the protein once said protein is extracted so as to reduce the perceived bitter taste therein.

The Applicant has already succeeded in providing other proteins with reduced bitterness, by modifying the protein extraction process. Thus, pea protein compositions with reduced bitterness, using a particular manufacturing process, have been disclosed in WO 2019/053387 A1.

The Applicant has also developed and disclosed, in application WO2017129921, other pea protein isolates of quite particular viscosity and solubility, the manufacturing process of which includes the use of proteolytic enzymes. This application also discloses food products comprising these isolates, in particular ready-to-drink beverages and ice creams, having a lower bitterness than those based on commercial pea protein isolates.

In the context of its studies for improving the taste of the protein, the Applicant observed that it was possible to reduce the bitterness of the legume proteins, and most particularly, the bitterness of pea proteins. The Applicant has identified that this objective of reducing bitterness could be achieved by implementing a process for extracting the legume protein wherein a phytic acid degrading enzyme is used. According to the invention, this introduction is used in particular locations of this extraction process. This method is particularly useful for providing legume proteins with more reduced bitterness that are useful for human food.

In the field of plant proteins, various documents disclose the use of enzymes degrading phytic acid. However, these enzymes are not used to influence the taste of these proteins and, particularly, to reduce the bitterness according to these documents.

For example, document U.S. Pat. No. 7,070,953 B1 discloses a process for producing a protein hydrolysate that can be less bitter wherein the hydrolysis is carried out with a proteolytic composition derived from Gadus species. If, among numerous other additional enzymes, a phytase can be mixed with this proteolytic composition, this phytase is not added in order to reduce the bitterness of the formed protein hydrolysate. Moreover, no example discloses the use of a phytase. Finally, this document does not disclose a process for extracting a legume protein.

Document WO2010/092778 A1 discloses the manufacture of hydrolyzed soy protein having a degree of solubility in trichloroacetic acid of less than 45% obtained by reacting a protease at a pH of less than 3.4, preferably in combination with heating at a temperature above 100° C. under acidic conditions. The phytase can be used in the process to increase the solubility of the soy protein under an acid pH. Similarly to document U.S. Pat. No. 7,070,953 B1, phytase is not added in order to reduce the bitterness of the formed protein hydrolysate.

Document US20070014909 A1 discloses the manufacture of an acidic beverage containing protein wherein the soy protein is introduced into the acidic beverage with a phytic acid degrading enzyme and the reaction is carried out, in-situ, in the acidic beverage. It is not disclosed to decrease the bitterness of the protein, compared to that obtained by a process which differs only in that it does not comprise this treatment step with a phytic acid degrading enzyme. In this document, treating the protein with phytic acid makes it possible to obtain a beverage with improved texture by increasing the solubility of the protein under an acid pH, in order to improve the mouthfeel as well as to limit the sedimentation of the proteins in the beverage.

WO95/27406 A1 discloses a method for treating a composition containing proteins, such as a de-oiled soy flour or soy flakes combining enzymes such as phytase, alpha-galactosidase and pectinases. Phytase is used to eliminate phytates that bind to minerals and prevent their absorption. The enzymes are not disclosed as reducing the bitterness of the protein-containing composition. Moreover, the improvement in the organoleptic properties of the ingredient is indicated in the document as being linked to better solubilization and a sweeter taste, which are explained by the hydrolysis of the pectic substances and of the galactooligosaccharides by the enzymes other than phytase, this hydrolysis generating sugars

The invention will now be disclosed below.

### SUMMARY OF THE INVENTION

The invention thus relates to a method for reducing the bitterness of a legume protein during a process for extracting said legume protein, said extraction process comprising:
preparing a suspension of legume flour
removing starch- and/or fiber-enriched fractions to form a liquid protein fraction
increasing the protein content of the liquid protein fraction to form a protein-enriched protein fraction
wherein the process comprises treating the liquid protein fraction and/or the protein-enriched protein fraction with a phytic acid degrading enzyme.

The invention also relates to the use of a phytic acid degrading enzyme in a legume protein extraction process to reduce the bitterness of said protein.

One advantage of the invention is that it does not require the addition of an additional compound such as a masking agent or a salt. Indeed, the Applicant has been able to observe that the phytic acid degrading enzymes, generally used to improve the digestibility of animal feed compositions, also make it possible to reduce the bitterness of a legume protein when they are used in steps of its extraction process. In the methods for reducing the bitterness where such additional compounds are mixed with the protein in order to reduce the bitterness thereof, it may then be necessary to label it, which tends to lengthen the list of components present in the ingredient and that of the final food product as well. Moreover, some of these additional compounds can be classified as additives according to certain regulations. However, consumers tend to prefer food products that are free of such additives. It is thus an additional advantage of this method to use a particular enzyme during the legume protein extraction process. Indeed, since this enzyme is a technological aid of the extraction process, its use has no impact on the labeling of the extracted legume protein.

The invention will now be disclosed in detail in the following paragraphs. The features disclosed may optionally be implemented. They may be implemented independently of one another or in combination with one another.

### DETAILED DESCRIPTION OF THE INVENTION

The invention relates to a method for reducing the bitterness of a legume protein during a process for extracting said protein.

“Reducing the bitterness of a legume protein” is understood to mean reducing the perception of a bitter flavor during tasting of the legume protein. The reduction in this perception can be determined according to the methods of the art, generally using a panel trained on tasting the legume protein as determined in the Examples section of the present Application.

The term “legume protein” should be understood in the present application to mean a composition extracted from leguminous plants comprising primarily polypeptide chains, or proteins, consisting of a sequence of amino acid residues bonded to one another by peptide bonds. The protein content of the legume protein is content N6.25, calculated using the Dumas method. Generally, the protein content is 60% or more, advantageously 80% or more, for example is 80% to 95%, especially 80 to 90%. According to the invention, protein isolate is understood to mean a legume protein having a protein content of 80% or more.

Although a legume protein is mainly defined by its botanical origin and its protein content, it obviously generally comprises other minority constituents other than proteins, such as starch, lipids, fibers, sugars and/or minerals. Generally, the total starch content in the legume protein produced according to the method of the invention ranges from 0% to 20%, for example from 0% to 10%, in particular from 0.5% to 5%. This total starch content can be measured using the AOAC 996.11 method. Generally, the total fiber content may range from 0% to 20%, for example from 1% to 18%, in particular from 2% to 10%. This content can be determined by the AOAC 2017.16 method. Generally, the total lipid content is from 0% to 15%, for example from 1% to 10%. The total lipid content can be determined by the AOAC 996.06 acid hydrolysis method. The sugar content may range from 0% to 10%, generally from 0.5% to 5%. The sugar content can be determined by high performance liquid chromatography (HPLC). The mineral content can be determined by determining the ash rate. Among the minerals, the legume protein comprises phosphates. The legume protein generally has a free phosphate content, which may vary widely, ranging from 0.2% to 2%, for example from 0.5% to 1.9%, in particular from 1.2% to 1.8%. The free phosphate content can be determined by anion chromatography. A person skilled in the art will know how to find the anionic chromatography conditions and, as an illustration, a chromatographic analysis method is indicated in the example section.

The legume protein obtained using the method according to the invention may comprise a phytic acid content varying from 0.1% to 1.5%, for example from 0.3% to 1.4% or even from 0.5% to 1.2%. According to the invention, the phytic acid content in the legume protein is determined according to the method disclosed in the document Mckie et al., Journal of AOAC International, Vol. 99, No. 3, 2016. This determination method is an indirect method consisting in quantifying the phosphate released during the hydrolysis of a sample by a phytase and an alkaline phosphatase. Thus, as explained in this document Mckie et al., it is specified that the determination of phytic acid according to this method uses the quantification of the phosphates released by the hydrolysis of phytic acid as well as the phosphates released by the hydrolysis of the ester derivatives of inositol IP1, IP2, IP3, IP4 and IP5 present in the sample. From this quantification of the released phosphate, an amount of phytic acid is deduced, which is an amount of theoretical phytic acid that would be obtained if all the inositol esters consisted of phytic acid. The company Megazyme markets the K-PHYT kit using this method.

“Leguminous plants” will be understood in the present application to mean the family of dicotyledonous plants of the Fabales order. This is one of the largest flowering plant families, third after Orchidaceae and Asteraceae in terms of number of species. It contains approximately 765 genera, bringing together more than 19,500 species. Several leguminous plants are significant crop plants, such as soybean, beans, in particular the mung bean, peas, chickpea, faba bean, groundnut, cultivated lentil, cultivated alfalfa, various clovers, broad beans, locust bean, licorice and lupin. Preferably, the leguminous plant is chosen from pea, faba bean and mung bean, most preferentially pea.

The term “pea” should be understood in the present application as all the wild varieties of “smooth pea” and all the mutant varieties of “smooth pea” and “wrinkled pea”.

In the invention, the process for extracting the legume protein comprises:
preparing a suspension of legume flour,
removing starch- and/or fiber-enriched fractions to form a liquid protein fraction,
increasing the protein content of the liquid protein fraction to form a protein-enriched protein fraction,
and this process comprises treating the liquid protein fraction and/or the protein-enriched protein fraction with a phytic acid degrading enzyme.

According to the invention, the reduction of the bitterness is observed for a protein obtained by the process above, comprising treating the liquid protein fraction and/or the protein-enriched protein fraction with a phytic acid degrading enzyme, in comparison with the protein obtained by a process that differs only in that it does not comprise this step of treatment with a phytic acid degrading enzyme. This improvement could be observed significantly, as demonstrated in the Examples section.

The protein extraction process comprises preparing a suspension of a legume flour.

The term “suspension of a legume flour” is understood to mean an aqueous suspension of leguminous plants milled in water.

Generally, the milled leguminous plants are milled seeds of leguminous plants. These seeds conventionally undergo prior steps that are well known to a person skilled in the art, such as, in particular, a cleaning or even the elimination of the outer shell from the seed (external cellulose husk), by a well-known step called “dehulling.”

Before milling, additional treatments of the seeds such as dry heating as disclosed in document WO2020/260841 can be carried out. Also, by wet route, a soaking or else a wet fermentation of a seed suspension as disclosed in document WO2015/071498 A1 can be applied.

The milling may for its part be carried out by dry milling, that is, by the passage of the dry seeds previously prepared in one or several mills. Alternatively, the milling may be wet milling, that is, by the passage of the seeds previously prepared in the form of a suspension of seeds in water in one or more mills.

Any type of suitable technology known to a person skilled in the art and adapted to the desired type of milling may be used as mill. It may in particular be ball mills, conical mills, helical mills, air jet mills, rotor-rotor systems or rotor-stator systems.

In the extraction process, a suspension of legume flour can thus be prepared by suspending a legume flour obtained by dry milling of the seeds in water. Alternatively, a suspension of legume flour can be prepared by wet milling. In this case, the suspension of legume flour can be obtained directly; the water present in the suspension can be preserved, but can also be renewed.

Preferably, the aqueous suspension of legume flour has between 15% and 25% by weight of dry matter (DM), preferentially about 20% by weight of DM, relative to the weight of said suspension.

In the present Application, the amounts by weight of the various constituents are expressed by dry weight.

At the end of milling, the pH can be checked. Preferably, the pH of the aqueous suspension is between 5.5 and 10, for example between 7 and 10, for example between 6 and 9, generally between 7 and 9. The pH may be adjusted by adding acid and/or base, organic or inorganic, for example sodium hydroxide or hydrochloric acid.

The process further comprises removing starch- and/or fiber-enriched fractions to form a liquid protein fraction. These starch- and/or fiber-enriched fractions mainly comprise the insoluble parts of the suspension. Thus, these fractions are conventionally recovered by known separation methods. It may in particular be carried out by means of at least one separation step with a decanter, in particular a centrifugal decanter, or else with hydrocyclones. The process can thus make it possible to recover one or more fractions fiber- and/or starch-enriched fractions that are removed from the suspension to form the liquid protein fraction.

The process further comprises a step of increasing the protein content.

According to a first variant of the extraction process, the step of increasing the protein content may comprise a stage of precipitation of the proteins comprised in the liquid protein fraction, followed by a separation stage to form the protein-enriched protein fraction. This stage can be carried out by bringing the pH of the aqueous suspension of legume flour closer to the isoelectric pH of the proteins to precipitate them. The precipitation stage may for example be carried out at a pH ranging from 4.6 to 5.4, for example approximately 5.0. This stage can also be carried out by thermocoagulating the proteins, that is, by heating the aqueous suspension of legume flour to a temperature ranging from 50° C. to 90° C. It is also possible to combine the two by carrying out this thermocoagulation after having brought the pH of the aqueous suspension of legume flour closer to the isoelectric pH of the proteins, in the above-mentioned pH ranges. The protein-enriched protein fraction can be obtained after a stage of separation of the precipitated proteins. This separation step can be carried out by filtration or by decanting, in particular by means of a centrifugal decanter. This variant is particularly preferred insofar as acid phosphatases, and in particular phytases, exhibit excellent activity at pH close to the isoelectric pH of the legume proteins; they are therefore highly active without the need to modify the pH of the liquid protein fraction or of the protein-enriched protein fraction.

According to a second variant of the extraction process, the step of increasing the protein content can be carried out by known membrane filtration methods. This membrane filtration step can thus in particular be ultrafiltration. Such processes using this method of increasing the protein content have already been disclosed, in particular in the document Fuhrmeister et al., Impact of processing on functional properties of protein products from wrinkled peas, Journal of Food Engineering 56 (2003) 119-129 or U.S. Pat. No. 10,143,226B1.

At the end of this step of increasing the protein content, the protein-enriched protein fraction may have the contents indicated in paragraphs [0029-0030] above for the legume protein, in proteins, fibers, starch, lipids, sugars and minerals.

According to the invention, the extraction process comprises treating the liquid protein fraction and/or the protein-enriched protein fraction with a phytic acid degrading enzyme. Since both variants are possible and the reaction conditions are similar in both cases, the terms “treating the protein fraction” encompass both variants of the treatment of the liquid protein fraction and/or of the protein-enriched protein fraction.

According to the invention, the term “phytic acid degrading enzyme” is understood to mean an enzyme capable of degrading phytic acid or phytates when used in an effective amount under its optimal usage conditions.

This enzyme can be a phosphatase, for example an acid phosphatase and more particularly a phytase. These enzymes are known to have the capacity to reduce the amount of phytic acid, which is an antinutritional factor preventing intestinal absorption of micronutrients such as iron, magnesium, zinc and copper. Phytic acid is mainly present in protein compositions of plant origin, in particular cereals and leguminous plants. Certain animals such as pigs and poultry are particularly sensitive to the presence of phytic acid or phytate in their feed. Thus today, these enzymes have still mainly been used today for animal nutrition, incorporating them as such in nutritional compositions for animals, in particular those intended for pigs and poultry. Alternatively, phytases have also been used by acting on proteins, in order to provide protein foods that are easier for the animals to absorb. To the Applicant's knowledge, while new phytases can now be used to provide ingredients intended for human food, there is still no commercially available legume protein manufactured by a process that uses such enzymes during its extraction process.

According to the invention, it is also possible to use several phytases (such as, for example, the mixture disclosed in document WO2007/006952 A1), several acid phosphatases or a mixture of acid phytase(s) and acid phosphatase(s).

Preferably, the phytic acid degrading enzyme is a phytase or a mixture of phytases. Phytases are hexakisphosphohydrolases that hydrolyze the phosphoester bonds present in phytic acid or phytates. Thus, they catalyze the hydrolysis of myo-inositol (1,2,3,4,5,6) hexakis phosphate into inorganic monophosphates and into myo-inositols phosphate of lower degree of phosphorylation (IP5 to IP1) and into free myo-inositol in certain cases.

There are three sub-classes of phytases, differentiated by the position of the first hydrolyzed phosphate. The 3-phytases (EC 3.1.3.8) hydrolyze the first phosphate in position 3, the 6-phytases (EC 3.1.3.26) hydrolyze the first phosphate in position 6, the 5-phytases (EC 3.1.3.72) hydrolyze the first phosphate in position 5.

The phytases can be obtained by a large variety of organisms: plants, animals, and especially microorganisms. These phytases can have different biochemical characteristics, in particular their activity based on pH and their temperature stability may differ.

Among the microorganisms producing phytases, mention will in particular be made of fungi, bacteria and yeasts.

The fungi may be of the genus Aspergillus, Penicillium, Mucor and Rhizopus. Among the fungi of the Aspergillus type, mention may be made of Aspergillus niger, Aspergillus oryzae, Aspergillus ficuum, Aspergillus awamori, Aspergillus nidulans and Aspergillus terreus and Trichoderma reesei.

The bacteria may be Pseudomonas sp., Klebsiella sp., Escherichia coli, Enterobacter sp., Bacillus sp., in particular Bacillus subtilis.

The yeasts are for example Saccharomyces cerevisiae, Candida tropicalis, Torulopsis Candida, Debaryomyces castellii, Debaryomyces occidentalis, Kluyveromyces fragilis and Peniophora lycii.

The phytases can also be extracted from plants, in particular can be endogenous phytases of cereals such as wheat, rye and barley.

According to the invention, the expression “a phytase that can be obtained” encompasses a phytase produced naturally by the particular strain and recovered from this strain and is encoded by a DNA sequence isolated from this strain and produced in a host organism transformed with said DNA sequence. The phytase can be obtained by fermentation of a microorganism producing phytase in a suitable nutrient medium, followed by the isolation of the phytase by methods known in the art. The medium used for the culture can be any medium suitable for culturing the microorganism in question. The medium preferably contains sources of carbon and nitrogen and other inorganic salts.

Preferred phytases useful for the invention are of the EC 3.1.3.8 type, for example SUMIZYME PHY, Shin Nihon and EC 3.1.3.26, for example NATUPHOS E 10000, BASF.

The phytic acid degrading enzyme can further comprise other enzymatic activities, such as for example secondary amylase activities, protease, cellulase, hemicellulase and/or pectinase activities.

The process can also use other enzymatic steps, for example steps of enzymatic proteolysis of the protein-enriched protein fraction. The enzymatic proteolysis is generally carried out in a known manner, conventionally using proteases. According to one embodiment, the extraction process comprises proteolysis of the protein-enriched protein fraction. According to one embodiment, the extraction process does not comprise proteolysis of the protein-enriched protein fraction. This step of proteolysis can make it possible to modify the degree of hydrolysis (DH) of the legume protein. The legume protein may have a degree of hydrolysis of less than 15%, advantageously less than 10%, preferably less than 6%, for example between 3% and 5%. A person skilled in the art will know how to adapt the enzymatic proteolysis conditions, or even will not carry out such a step in order to obtain the desired DH.

According to the invention, the treatment on a protein fraction comprises introducing at least one phytic acid degrading enzyme into said treated fraction and the action of the enzyme in order to reduce the amounts of phytic acid. The enzyme introduced is generally in the form of an enzymatic solution. According to one embodiment, the amount of phytic acid degrading enzyme, expressed in dry mass relative to the dry mass of the protein fraction, ranges from 0.1% to 3%, for example from 0.2% to 1%. It is possible to dilute or concentrate the treated protein fraction using the known concentration and dilution methods. One of the advantages of the method of the invention is that it is also possible not to modify the dry matter of the protein fraction and to introduce the enzyme directly therein. Generally, during the action of the enzyme, the mass of dry matter of the protein fraction during the treatment step with the phytic acid degrading enzyme ranges from 3% to 30%, for example from 4% to 20%, for example between 5% and 15%. Generally, during the action of the enzyme, the pH of the protein fraction is from 4 to 7, for example from 4.5 to 6, in particular from 4.6 to 5.4, for example about 5.0. The duration of the treatment may also vary; it may range from a few minutes to a few hours, for example from 10 to 240 minutes, in particular from 15 to 100 minutes, generally from 20 to 80 minutes.

The conditions concerning pH, temperature, dry matter, quantity of enzyme and time will be easily adapted by a person skilled in the art so that the enzyme is effective: according to the invention, the treatment step is carried out so as to reduce the quantities of phytic acid in the protein fraction. Preferably, the treatment step with the phytic acid degrading enzyme is carried out until the phytic acid content in the treated fraction is less than 1% by mass relative to the dry mass of this fraction.

By way of illustration, according to the first variant of the extraction process where the step of increasing the protein content comprises a protein precipitation stage followed by a separation stage, the step of treating the liquid protein fraction with a phytic acid degrading enzyme can be carried out before, during or after having brought the pH of the aqueous suspension of legume flour closer to the isoelectric pH. Preferably, the treatment step is carried out simultaneously or afterwards, most preferentially simultaneously. This embodiment is shown in Example 4 below.

Alternatively, still according to this same first variant, the step of treating the protein-enriched protein fraction with a phytic acid degrading enzyme can be carried out on the protein-enriched protein fraction recovered during the separation stage, optionally after dilution of the latter. This embodiment is in particular shown in Example 2 below.

It is possible to resolubilize the recovered protein-enriched protein fraction, and then to centrifuge it. This step makes it possible to wash the protein and to eliminate compounds other than proteins, such as salts. However, this step is not necessary and according to a preferred embodiment of the method of the invention, the extraction process does not comprise steps of resolubilizing and centrifuging the protein-enriched protein fraction.

The process may further comprise, after the treatment step with the phytic acid degrading enzyme, a step of heat treatment of the protein-enriched protein fraction. This step is carried out to ensure satisfactory bacteriological quality of and/or to modify the functionalities of the legume protein. This step can be carried out directly after the recovery of the protein-enriched protein fraction. This heat treatment can be carried out at a temperature ranging from 75° C. to 160° C. and for 0.1 seconds to 30 minutes. Advantageously, this heat treatment may also be used to functionalize the protein composition. It is therefore preferentially performed with a conventional protocol of 100° C. to 160° C. for 0.01 s to 15 s, preferentially between 1 and 2 seconds, immediately followed by cooling.

The process may also further comprise a step of drying the protein-enriched protein fraction. Generally, this drying step is carried out so as to reach a dry matter content of greater than 80%, preferentially greater than 90% by weight of dry matter relative to the weight of said protein-enriched protein fraction. To this end, any technique well known to those skilled in the art can be used, for instance freeze-drying, flash drying or drying on a drying cylinder, or atomization. The process may also comprise a step of milling or micronizing. Atomization is the preferred technology, in particular multiple-effect atomization. The legume protein may be in the form of a powder having a particle size d50, which can vary widely, for example from 10 μm to 200 μm.

Generally, the resulting legume protein can be used in food products and beverages that may include it in an amount up to 100% by weight relative to the total dry weight of the food or beverage product, for example in an amount ranging from about 1% by weight to about 80% by weight relative to the total dry weight of the food or beverage product. All intermediate amounts (that is, 2%, 3%, 4% . . . 77%, 78%, 79% by weight relative to the total weight of the food or beverage product) can be used, as well as all the intermediate ranges based on these quantities. The food or beverage products that can be concerned comprise baked products; sweet baked products (including, but not limited to, rolls, cakes, pies, pastries, and cookies); pre-made sweet baking mixtures for preparing sweet baked products; pie fillings and other sweet fillings (including, but not limited to, fillings for fruit pies and fillings for nut pies, such as fillings for pecan pies, as well as fillings for cookies, cakes, pastries, confectionery products and similar products, such as fillings for fat-based cream); desserts, gelatins and puddings; frozen desserts (including, but not limited to, frozen dairy desserts such as ice cream-including ordinary ice cream, soft serve ice cream and all other types of ice cream- and non-dairy frozen desserts such as non-dairy ice cream, sorbet and similar products); carbonated beverages (including, but not limited to, carbonated soft drinks); non-carbonated beverages (including, but not limited to, non-carbonated soft drinks such as flavored waters, fruit juices and sweetened tea or coffee-based beverages); beverage concentrates (including, but not limited to, liquid concentrates and syrups as well as non-liquid “concentrates,” such as freeze-dried and/or powdered preparations); yogurts (including, but not limited to, high-fat, reduced-fat and fat-free dairy yogurts, as well as non-dairy and lactose-free yogurts); snack bars (including, but not limited to, cereal bars, nut bars, and/or fruit bars); bread products (including, but not limited to, yeasted and unyeasted breads, yeasted breads and undyed breads such as soda breads, breads comprising any type of wheat flour, breads composed of any type of non-wheat flour (such as potato, rice and rye flour), gluten-free bread); bread mixtures for preparing bread products; sauces, syrups and vinaigrettes; sweetened spreads (including, but not limited to, jellies, jams, butters, nut spreads and other preserves, preserves and other spreadable preserves); confectionery products (including, but not limited to, jelly beans, soft candies, hard candies, chocolates and gums); sugar-coated and not sugar-coated breakfast cereals (including, but not limited to, extruded breakfast cereals, flaked breakfast cereals and blown breakfast cereals); and coating compositions for cereals intended for the preparation of sweet breakfast cereals. Other types of food and beverages that are not mentioned herein but which conventionally comprise one or more proteins can also be envisaged in the context of the present invention. In particular, animal feed (such as pet food) is explicitly envisaged. It can also be used, optionally after texturing by extrusion, in products similar to meat such as emulsified sausages or plant-based hamburgers. It can also be used in egg replacement formulations.

The food or beverage product can be used in specialized nutrition, for specific populations, for example for babies or infants, elderly people, athletes, or in clinical nutrition (for example, feeding by probe or enteral nutrition).

The legume protein can be used as a single source of proteins, but can also be used in combination with other plant or animal proteins. The term “plant protein” denotes all the proteins derived from cereals, oleaginous plants, leguminous plants and tuberous plants, as well as all the proteins derived from algae and microalgae or fungi, used alone or as a mixture, selected from the same family or from different families. In the present application, the term “cereals” refers to plants cultivated from the family of grasses producing edible grains, for example wheat, rye, barley, corn, sorghum or rice. Grain are often ground in flour form, but are also provided in the form of cereals and sometimes in the form of whole plants (forage crops). Tubers may be carrot, cassava, konjac, potato, Jerusalem artichoke, sweet potato. The animal protein can, for example, be egg or milk proteins, such as whey proteins, casein or caseinate proteins. The pea protein composition can thus be used in association with one or more of these proteins or amino acids in order to improve the nutritional properties of the final product, for example to improve the PDCAAS of the protein or to provide or modify other functionalities.

The resulting legume protein has improved organoleptic properties and in particular reduced bitterness compared to the same legume protein that was not prepared by the method according to the invention comprising the treatment of the liquid protein fraction and/or of the protein fraction enriched with a phytic acid degrading enzyme. Another observed advantage is that the solubility in water under an acid pH, for example between 3 and 5 and in particular at pH 4, is increased, as demonstrated in the examples section. This solubility at pH 4 may range from 26% to 50%, for example from 30% to 45%. The method for determining the solubility is disclosed in the examples section below. The legume protein of the invention can thus be used in beverages under an acid pH, which will have an improved texture.

The invention will be better understood with the aid of the following examples, which are intended to be illustrative and non-limiting.

### EXAMPLES

### Methods

### Evaluation of the Bitterness

The organoleptic study of the different pea protein powders obtained is carried out with the help of a panel and following the protocol hereunder.

The panel consists of 30 persons with 2 to 4 years of training. Their performance is frequently verified in terms of sensitivity, consensus and repeatability.

The tasting matrices consist of suspensions of each of the powders at 4% by weight in Evian® water, homogenized using an immersion mixer.

The tasting conditions are as follows: individual stall, white walls, calm atmosphere, red light, late morning, products coded with 3 digits, presented in a random order, and use of apple and/or water to rinse the oral cavity.

The methodology used is called “BLOCK PROFILING”. This method is referred to as Quantitative Descriptive Analysis (QDA): the panelists score each product on a scale of intensity (from 0 to 10) through different indicators that correspond, for example, to tastes, flavors, or particular notes.

The control is always presented first and is presented blindly in 1 out of 2 sessions.

The panelists conduct the tasting exercise in blocks: they evaluate each product individually (starting with the control) in a first block (indicators: salty, bitter, astringent, sandy—with a nose clip), then they analyze all the products in a second block (indicators: pea, broth, walnut, almond). Finally, they repeat the exercise in a third block (indicators: potato, cereal). The products are evaluated in multiple sessions, until reaching 10 evaluations. The arithmetic average of these 10 evaluations is then calculated for each indicator. The bitterness reduction is calculated for the tests according to the invention (2, 4 and 6) according to the following formula: (Control Score−Invention Sample Score)/Control Score×100.

### Protein Content:

Percentage by dry mass of protein N6.25 using the Dumas method (ISO 16634).

### Determining the Content of Phytic Acid:

The phytic acid content is determined by the Megazyme K-PHYT kit according to the method disclosed in the document Mckie et al., Journal of AOAC International, Vol. 99, No. 3, 2016.

### Determining the Content of Free Phosphate:

The free phosphate content is determined according to the anionic chromatography method known in the art. The legume protein sample is analyzed, according to the guidelines in the user's manual, on a Thermo ICS-2100 chromatograph equipped with an AG 11 HC DIONEX pre-column and an AS 11 HC DIONEX column, combined with a conductometric detector, using a solution of potassium hydroxide as eluent.

### Measuring the Solubility in Water at Different pH

This measurement is based on the dilution of the sample in distilled water, its centrifuging and the analysis of the supernatant.

### Procedure:

Introduce 150 g of distilled water into a 400 ml beaker at 20° C.+2° C., mix with a magnetic stirrer bar, and add precisely 5 g of the sample to be tested.

Adjust the pH to the desired value with 0.1 N NaOH or HCl (pH 4 or pH 7), or do not adjust it.

Complete water content at 200 g.

Mix for 30 minutes at 1000 rpm and centrifuge for 15 minutes at 3000 g.

Collect 25 g of the supernatant.

Introduce into a previously dried and tared crystallizer.

Place in an oven at 103° C.±2° C. for 1 hour.

Then place in a desiccator (with desiccant) to cool to ambient temperature and weigh.

The soluble dry matter content, expressed in % by weight, is given by the following formula:

$$\frac{( m 1 - m 2 ) \times ( 200 + P ) \times 100}{P 1 \times P} = \% solubility$$

wherein:
   P=weight, in g, of the sample=5 g
   m1=weight, in g, of the crystallizing dish after drying
   m2=weight, in g, of the empty crystallizing dish
   P1=weight, in g, of the sample collected=25 g

### Example 1 (Control)

The following protocol is applied:
Dilution of 2 kg (expressed in commercial mass) of yellow pea flour (86% dry matter and 27% protein N6.25 by dry weight) with demineralized water (20° C.)=>Suspension at 17% solids with a RAYNERI-type bench stirrer with a dispersion turbine for 30 minutes
Centrifugation 1000 g—5 minutes/Beckman centrifuge
Recovery of 7 kg of supernatant at about 7% dry matter in a stainless steel container and removal of the fiber/starch pellet
Acidification=>pH 5 with 1N HCl
Thermo-flocculation: Heating and holding for 10 minutes at 60° C. (heating plate) with stirring
Centrifugation 5000 g—5 minutes/BECKMAN centrifuge
Recovery of the protein sediment (or protein-enriched protein fraction) and removal of the supernatant
Dilution of sediment with demineralized water (20° C.)=>3 kg of suspension at 12% dry matter with a RAYNERI-type bench stirrer with dispersion turbine for 30 minutes
Neutralization (under stirring)=>pH7 with 1N NaOH
Heat treatment-type pasteurization: heating=>80° C. (in stirred crystallizing dish)+holding for 3 minutes
Spray drying with a BIICHI atomizer

### Example 2

The protocol of this test, which mainly differs from the control protocol by the use in the process for extracting a phytase before the neutralization step, is applied. Thus, this test is identical to the one of example 1 except in that between the step of diluting the sediment and the neutralization step, the following additional steps are carried out:
Heating=>55° C. on stirred heating plate

Introduction of 0.5% by dry weight of phytase relative to the dry weight of suspension (SUMIZYME PHY, Shi Nihon), or an enzymatic activity of 3600 FTU according to the supplier's data, in 3 kg of suspension at 12% dry matter and hydrolysis with stirring for 20 minutes.

### Example 3 (Comparative Example)

This test aims to evaluate the impact of the use of the SUMIZYME PHY enzyme and of the heat treatment used during hydrolysis on the properties of the protein. The protocol of example 3 thus differs from the one of example 2 only in that no phytase is introduced after the diluted sediment is heated at 55° C. Thus, the dried protein of example 3 has exactly the same thermal history as the dried protein in example 2.

### Example 4

The protocol of this test differs from the protocol of example 2 mainly in that the phytase is introduced into the extraction process after the step of removing the fibers and the starch rather than after the step of protein sediment dispersion. Thus, this test is identical to the one of example 1 except in that between the acidification step and the thermo-flocculation step, the following additional steps are carried out:
Heating the acidified supernatant=>55° C. on stirred heating plate

Introduce 0.5% by dry weight of phytase relative to the dry weight of supernatant and hydrolysis with stirring for 20 minutes.

The invention was also evaluated on a larger scale (pilot scale) in the following examples 5 and 6.

### Example 5-Pilot Scale Control

The control protocol below was carried out:
Dilution of 80 kg by dry weight of smooth yellow pea flour with demineralized water (20° C.)=>Suspension at 15% solids in a tank equipped with axial and peripheral stirrer blades for 30 minutes
Decanting in a Flottweg tank type Z2—4000 g
Transfer of the supernatant (approximately 7% of dry matter) in stirred tank equipped with a double jacket and removal of the fiber/starch pellet
Acidification=>pH 5 with HCl
Thermo-flocculation: heating and holding for 10 minutes at 60° C. in a double jacketed tank heated by steam
Decanting in a Flottweg tank type Z2-4000 g
Recovery of the protein sediment and removal of the supernatant
Dilution of sediment with demineralized water (20° C.) in stirred tank=>Suspension at 12% solids with propeller stirrer for 30 minutes
Neutralization (under stirring)=>pH7 with 1N NaOH
Heat treatment by direct steam injection for 10 s at 135° C., then flash at 70° C.
Spray drying in a NUBILOSA pilot atomizer: drying temperature 190-195° C.; product outlet temperature 90-95° C.

### Example 6-Pilot Scale Test

The protocol below, which differs from the protocol of example 5 mainly by the use of a phytase in the extraction process before the neutralization step. Thus, this test is identical to the one of example 5 except in that between the step of diluting the protein sediment and the neutralization step, the following additional steps are carried out:
Heating=>55° C. on a stirred heating plate in stirred tank with double jacket
Introduction of 0.5% by dry weight of phytase relative to the dry weight of suspension, or 30,000 FTU, in 25 kg of suspension at 12% dry matter and hydrolysis with stirring for 20 minutes

Properties of the proteins obtained in examples 1 to 6:

The properties of the products obtained are shown in the table below:

| | | Protein | | | | | |
|---|---|---|---|---|---|---|---|
| | | content | Phytic | Free | | | Bitterness |
| | | N6.25 | acid | phosphate | Solubility | Solubility | reduction |
| | | % DM | content | content | pH4 (%) | pH7 (%) | (%) |
| LAB | Example 1 | | 2.1 | 0.07 | 26 | 54 | |
| | Example 2 | | 0.8 | 1.6 | 40 | 40 | 38% |
| | Example 3 | | NM | NM | 18 | 51 | |
| | Example 4 | | 0.4 | 0.6 | 39 | 47 | 24% |
| pilot | Example 5 | 87.6 | 2.0 | 0.07 | 15 | 70 | |
| | Example 6 | 87.9 | 0.4 | 1.1 | 27 | 30 | 26% |

The tests demonstrate that the use of a phytase in the extraction process makes it possible to reduce the bitterness of the formed protein, in comparison with a process that does not comprise such a treatment.

In the variant where the treatment step with a phytase is carried out on the protein fraction, the phosphate content is more reduced.

The protein further has an improved acid pH solubility in the tests comprising the treatment with a phytase.

Other tests were carried out with a NATUPHOS E 10000 phytase, BASF, instead of the SUMIZYME PHY and similar results were obtained.

1. A method for reducing the bitterness of a legume protein during a process for extracting said legume protein, said extraction process comprising:
preparing a suspension of legume flour;
removing starch- and/or fiber-enriched fractions to form a liquid protein fraction; and,
increasing the protein content of the liquid protein fraction to form a protein-enriched protein fraction, wherein the process comprises treating the liquid protein fraction and/or the protein-enriched protein fraction with a phytic acid degrading enzyme.

2. The method according to claim 1, wherein in the step of increasing the protein content may comprise a stage of precipitation of the proteins comprised in the liquid protein fraction, followed by a separation stage to form the protein-enriched protein fraction.

3. The method according to claim 2, wherein the precipitation stage is carried out under a pH ranging from 4.6 to 5.4, for example about 5.0.

4. The method according to claim 1, wherein the step of increasing the protein content comprises membrane filtration.

5. The method according to claim 1, wherein the extraction process comprises a step of treating the liquid protein fraction with a phytic acid degrading enzyme.

6. The method according to claim 1, wherein the extraction process comprises a step of treating the protein-enriched protein fraction with a phytic acid degrading enzyme.

7. The method according to claim 1, wherein the extraction process further comprises a step of proteolysis of the protein-enriched protein fraction.

8. The method according to claim 1, wherein the extraction process does not comprise proteolysis of the protein-enriched protein fraction.

9. The method according to claim 1, wherein the phytic acid degrading enzyme is a phytase.

10. The method according to claim 1, wherein the process further comprises, after the treatment step with the phytic acid degrading enzyme, a step of heat treatment of the protein-enriched protein fraction.

11. The method according to claim 1, wherein the mass percentage of dry matter of the protein fraction during the treatment step with the phytic acid degrading enzyme is from 4% to 20%, in particular from 5% to 15%.

12. The method according to claim 1, wherein the treatment step with the phytic acid degrading enzyme is carried out until the phytic acid content in the treated fraction is less than 1% by mass relative to the dry mass of this fraction.

13. The method according to claim 1, wherein the legume protein has a degree of hydrolysis of less than 15%, advantageously less than 10%, preferably less than 6%, for example between 3% and 5%.

14. The method according to claim 1, wherein the amount of phytic acid degrading enzyme, expressed in dry mass relative to the dry mass of the liquid protein fraction or of the protein-enriched protein fraction, ranges from 0.1% to 3%, for example from 0.2% to 1%.

15. The method according to claim 1, wherein the process further comprises a step of drying the protein-enriched protein fraction.

16. The method according to claim 1, wherein the leguminous plant is chosen from pea, faba bean and mung bean, most preferentially pea.

17. A use of a phytic acid degrading enzyme in a legume protein extraction process to reduce the bitterness thereof.

18. The use according to claim 17, wherein the leguminous plant is chosen from pea, faba bean and mung bean, most preferentially pea.
